(19) United States
(12) Patent Application Publication
(10) Pub. No.: US 2020/0261419 A1
(43) Pub. Date: Aug. 20, 2020

(54) COMBINATIONS FOR THE TREATMENT OF KIDNEY STONES

(71) Applicant: Wake Forest University Health Sciences, Winston-Salem, NC (US)

(72) Inventors: W. Todd Lowther, Pfafftown, NC (US); Ross P. Holmes, Birmingham, AL (US); Daniel Yohannes, Winston-Salem, NC (US)

(73) Assignee: Wake Forest University Health Sciences, Winston-Salem, NC (US)

(21) Appl. No.: 15/781,600

(22) PCT Filed: Dec. 7, 2016
(86) PCT No.: PCT/US2016/065305

§ 371 Date: Jun. 5, 2018

Related U.S. Application Data
(60) Provisional application No. 62/264,020, filed on Dec. 7, 2015.

(51) Int. Cl. A61K 31/4164; A61K 31/415; A61K 9/00; A61K 9/20; A61K 9/48; A61P 13/04; A61K 31/4192

(52) U.S. Cl. CPC A61K 31/4164; A61K 31/415; A61K 9/0056; A61K 31/4192; A61K 9/4808; A61P 13/04; A61K 9/20

(57) ABSTRACT

Provided herein are methods of treatment for kidney stones, e.g., for controlling or inhibiting the formation of calcium oxalate kidney stones by inhibiting the production of glyoxylate and/or oxalate, treatment of primary hyperoxaluria, etc. In some embodiments, methods comprise administering to a subject in need thereof, in combination, an inhibitor of hydroxyproline dehydrogenase (HYPDH), an inhibitor of glycolate oxidase (GO), and/or another agent for the treatment of kidney stones. Compositions for such use or the use of active agents in the manufacture of a medicament for the treatment of kidney stones are also provided.

### RELATED APPLICATIONS

This application claims the benefit of U.S. Provisional Patent Application Ser. No. 62/264,020, filed Dec. 7, 2015, the disclosure of which is incorporated by reference herein in its entirety.

### GOVERNMENT FUNDING

This invention was made with government support under grant numbers DK083527 and DK073732 awarded by National Institutes of Health. The United States government has certain rights in the invention.

### BACKGROUND

Kidney stones affect approximately 1 in 11 individuals in the United States. The 2012 National Health and Nutrition and Examination Survey (NHANES), part of the Urological Diseases in America Project, reported that the overall prevalence of kidney stones was 8.8% (10.6% and 7.1% for men and women, respectively) (Jiang et al., Am J Physiol Gastrointest Liver Physiol 302, G637-643, 2012). This study and others attest to the significant increase in stone cases in general, but especially in individuals with obesity, diabetes, and following bariatric surgery (Jiang et al., supra; Knight et al., Am J Nephrol 25, 171-175, 2005). The direct and indirect costs associated with kidney stone treatment (i.e., nephrocalcinosis) are significant (Knight et al., Kidney Int 70, 1929-1934, 2006).

Individuals with Primary Hyperoxaluria (PH) have mutations in a variety of genes involved in glyoxylate and hydroxyproline (Hyp) metabolism that result in a significant increase in oxalate production and deposition of calcium oxalate stones, the most common type of stones for all stone formers. The treatments for these individuals range from a combined kidney-liver transplant to a life-long use of potassium citrate, increased fluid intake and dietary restriction of oxalate (Riedel et al., PLoS One 6, e26021, 2011; Knight et al., Am J Physiol-Renal 302, F688-693, 2012). Treatments for the removal of stones currently include shock-wave lithotripsy, ureteroscopic stone removal, and percutaneous nephrolithotomy (Riedel et al., supra). However, the recurrence of stones following the available procedures is over 50%.

Kidney stones are also a significant problem in veterinary medicine. Pets such as dogs and cats can develop stones that lead to painful urination and/or a life-threatening blockage.

Considering that the current treatments only address symptoms, novel treatments to prevent the formation of stones in PH and other idiopathic stone formers are greatly needed.

### SUMMARY

Provided herein are methods of treating kidney stones (e.g., controlling or inhibiting the formation of oxalate kidney stones; treating primary hyperoxaluria), comprising administering to a subject in need thereof, in combination, a hydroxyproline dehydrogenase (HYPDH) inhibitor, a glycolate oxidase (GO) inhibitor, and/or another agent for the treatment of kidney stones.

In some embodiments, the HYPDH inhibitor is a compound of Formula I, a compound of Formula II, or a compound of Formula III:

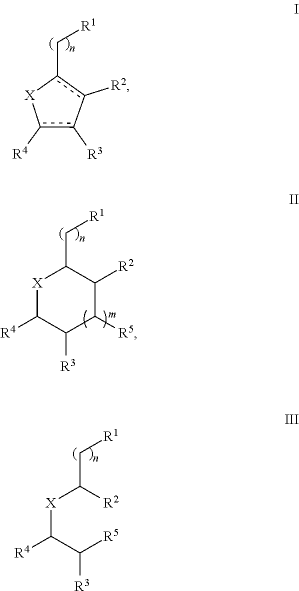

wherein:

X is O, S, NH, NMe or C^{x}R^{y}, wherein R^{x} and R^{y} are each independently selected from H, alkyl or halo;

n is 0, 1, 2, 3, 4, 5 or 6;

m is 0, 1, 2, or 3;

R¹ is selected from the group consisting of: alkyl, alkenyl, alkynyl, aryl, halo, hydroxy, amine and carboxy;

R² is selected from the group consisting of: H, alkyl (e.g., lower alkyl), hydroxy, amine, and ═O; or R² is R^{2a}R^{2b}, wherein R^{2a} and R^{2b} are each independently selected from alkyl (e.g., lower alkyl) and hydroxy;

R³ is selected from the group consisting of: H, hydroxy, amine, and ═O; or R³ is R^{3a}R^{3b}, wherein R^{3a} and R^{3b} are each independently selected from alkyl (e.g., lower alkyl) and hydroxy;

R⁴ is selected from the group consisting of: H, alkyl (e.g., lower alkyl), and hydroxy; or R⁴ is R^{4a}R^{4b} wherein R^{4a} and R^{4b} are each independently selected from alkyl (e.g., lower alkyl), hydroxy, and halo, wherein said alkyl may be unsubstituted or substituted 1, 2 or 3 times with hydroxy; and

each R⁵ is independently selected from the group consisting of: H, alkyl (e.g., lower alkyl), hydroxy, amine, and ═O; or R⁵ is R^{5a}R^{5b} wherein R^{5a} and R^{5b} are each independently selected from alkyl (e.g., lower alkyl) and hydroxy; or R² and an adjacent R⁵ are taken together to form an aryl or heteroaryl,

or a pharmaceutically acceptable salt or prodrug thereof.

In some embodiments, the HYPDH inhibitor is a compound of Formula I:

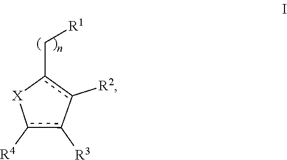

wherein:

X is S;

n is 0;

R¹ is selected from the group consisting of: alkyl, alkenyl, alkynyl, aryl, halo, hydroxy, amine and carboxy;

R² is selected from the group consisting of: H and lower alkyl;

R³ is selected from the group consisting of: hydroxy, amine, and ═O; or R³ is R^{3a}R^{3b}, wherein R^{3a} and R^{3b} are each independently selected from alkyl (e.g., lower alkyl) and hydroxy;

R⁴ is selected from the group consisting of: H and lower alkyl;

or a pharmaceutically acceptable salt or prodrug thereof.

In some embodiments, the GO inhibitor is a compound of Formula IV or Formula V:

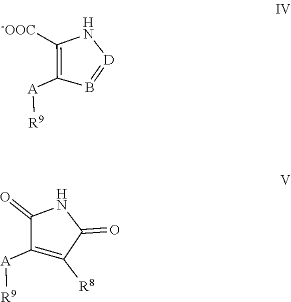

wherein:

A is CH₂ or S;

B is CH or N;

D is CH or N;

R⁸ is H or OH; and

R⁹ is aryl or heteroaryl, wherein said aryl or heteroaryl has two aromatic rings, which rings are fused or directly adjoining,

or a pharmaceutically acceptable salt or prodrug thereof.

In some embodiments, the GO inhibitor is a compound of Formula IV:

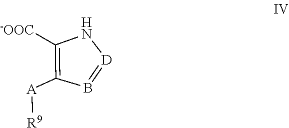

wherein:

A is CH₂ or S;

B is CH or N;

D is CH or N; and

R⁹ is aryl or heteroaryl, wherein said aryl or heteroaryl has two aromatic rings, which rings are fused or directly adjoining,

or a pharmaceutically acceptable salt or prodrug thereof.

In some embodiments, the R⁹ is selected from the group consisting of:

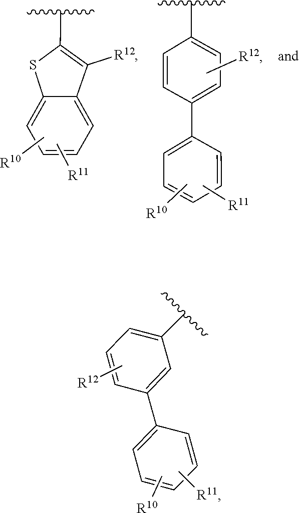

wherein R¹⁰, R¹¹ and R¹² are each independently selected from the group consisting of: H, alkyl, halo and haloalkyl,

or a pharmaceutically acceptable salt or prodrug thereof.

In some embodiments, the HYPDH inhibitor is administered in combination with said GO inhibitor.

In some embodiments, the method comprises administering a diuretic, a calcium oxalate crystallization inhibitor, an AGT cofactor or a kidney sodium glucose transporter inhibitor in combination with said HYPDH inhibitor and/or GO inhibitor.

Also provided are pharmaceutical compositions comprising an HYPDH inhibitor, a GO inhibitor and/or another inhibitor of kidney stone formation. In some embodiments, the composition is formulated for oral administration. In some embodiments, the composition is a food product formulation.

Also provided is the use of an HYPDH inhibitor, a GO inhibitor and/or another agent for the treatment of kidney stones in combination for treating kidney stones (e.g., inhibiting the formation of oxalate kidney stones; treating primary hyperoxaluria) in a human or non-human animal subject in need thereof.

Further provided is the use of an HYPDH inhibitor, a GO inhibitor and/or another agent in the preparation of a medicament for treating kidney stones (e.g., inhibiting the formation of oxalate kidney stones; treating primary hyperoxaluria) in combination as taught herein, in a human or non-human animal subject in need thereof.

### BRIEF DESCRIPTION OF THE DRAWINGS

FIG. 1 presents a schematic of the metabolism of 4-hydroxyproline, glycolate and glyoxylate within a hepatocyte. Four mitochondrial enzymes are responsible for Hyp breakdown: hydroxyproline dehydrogenase (HYPDH), Δ¹-pyrroline-5-carboxylate dehydrogenase (1P5CDH), aspartate aminotransferase (AspAT), and 4-hydroxy-2-oxoglutarate aldolase (HOGA). A variety of enzymes, including alanine-glyoxylate aminotransferase (AGT), D-amino acid oxidase (DAO), glyoxylate reductase (GR), and lactate dehydrogenase (LDH), can act on the glyoxylate produced from HOG cleavage. AGT, GR, and HOGA are mutated within primary hyperoxaluria patients (PH type 1, 2, and 3, respectively). Glycolate oxidase (GO) can readily convert glycolate back into glyoxylate within the peroxisome; a feature that is particularly problematic for PH2 patients.

FIG. 2 presents the structures of Hyp analogs, of which some have been tested for HYPDH inhibition.

### DETAILED DESCRIPTION

Provided herein are methods of treatment for kidney stones, e.g., controlling or inhibiting the formation of kidney stones, comprising administering to a subject in need thereof, in combination, an inhibitor of hydroxyproline dehydrogenase (HYPDH), an inhibitor of glycolate oxidase (GO), and/or another active agent for the treatment of kidney stones.

In some embodiments, the HYPDH inhibitor and GO inhibitor combination treatment beneficially results in an additive and/or synergistic effect in the control or inhibition of kidney stone formulation. In some embodiments, the HYPDH inhibitor and/or GO inhibitor may beneficially act at both the liver and the kidney sites of hydroxyproline metabolism.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

“Subject” or “patient” as used herein are generally mammalian subjects, including both human subjects and non-human mammalian subjects (e.g., dog, cat, horse, etc.) for research or veterinary purposes. Subjects may be male or female and may be of any suitable age, including neonate, infant, juvenile, adolescent, adult, and geriatric subjects.

“Treat” as used herein refers to any type of treatment that imparts a benefit to a subject, particularly slowing or inhibiting the formation of glyoxylate and/or oxalate, decreasing urinary oxalate, slowing or inhibiting the formation of calcium oxalate stones in the kidneys and/or urinary tract (kidneys, ureters, bladder, and urethra), and/or the deposition of calcium oxalate in other tissues such as the heart. For example, the treatment may reduce the size of and/or decrease the number of such stones, inhibit or slow the growth of such stones or calcium oxalate deposition in tissues such as the heart, alleviate symptoms of such stones or deposition, etc. Treatment may also include prophylactic treatment of a subject deemed to be at risk of kidney stone formation (e.g., after bariatric surgery and recurrent idiopathic stone formers).

“Kidney stones” are hard deposits of minerals that form a stone or crystal aggregation, which may result in damage or failure of the kidney and/or urinary tract function. Most kidney stones are calcium stones, usually in the form of calcium oxalate.

“Oxalate” or “oxalic acid” is a dianion of the formula C₂O₄²⁻ produced by the body and also commonly ingested in the diet. Oxalate can combine with calcium in the kidneys or urinary tract to faun calcium oxalate, which is the main component of most kidney stones.

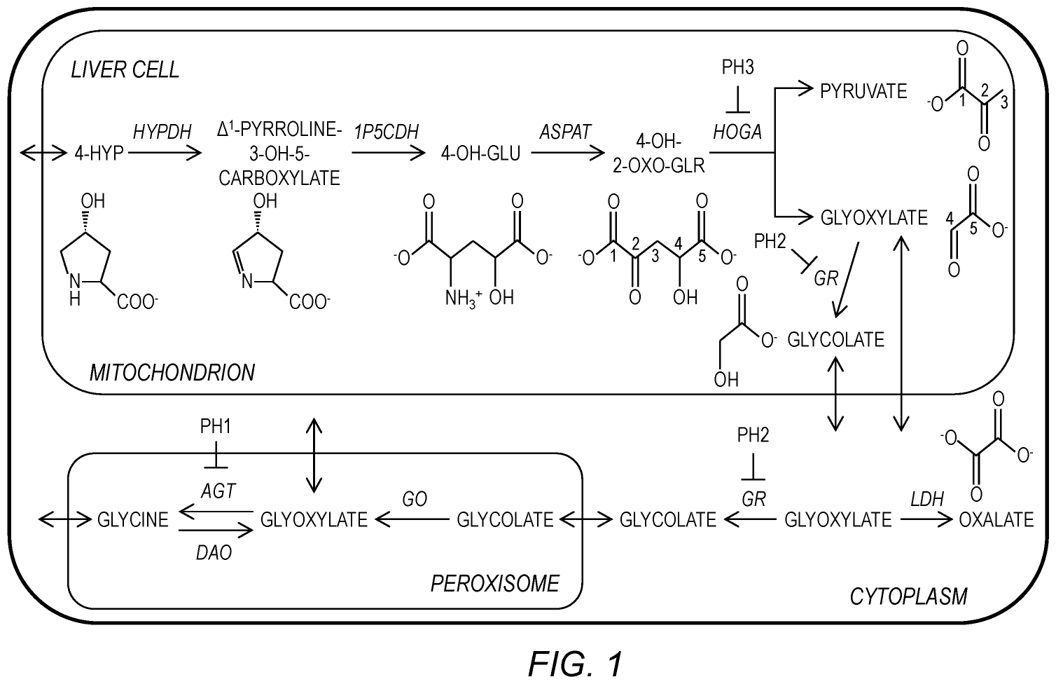

“Glyoxylate” is a precursor of oxalate, as shown in FIG. 1.

“Glycolate oxidase” or “GO” is an enzyme that catalyzes the oxidation of glycolate. Multiple GO isoforms exist, such as GO1 (predominantly in liver) and GO2 (located in kidney and liver) (Jones et al. J Biol Chem 275, 12590-12597, 2000). GO1 catalyzes the FMN-dependent oxidation of glycolate to glyoxylate, and glyoxylate to oxalate, although the latter occurs with a 100-fold lower kcat/Km value (Murray et al. Biochemistry 47, 2439-2449, 2008).

“Primary hyperoxaluria” is a condition characterized by the overproduction of oxalate and/or defective production or function of one or more enzymes that regulate the levels of oxalate in the body. Sufferers of Type 1 primary hyperoxaluria have a defect or shortage of the alanine:glyoxylate aminotransferase enzyme (AGT). Type 2 primary hyperoxaluria sufferers have a defect or shortage of the glyoxylate reductase enzyme (GR). Type 3 primary hyperoxaluria sufferers have a defect or shortage of the 4-hydroxy-2-oxoglutarate aldolase (HOGA).

“Hydroxyproline” or “Hyp” has the structure:

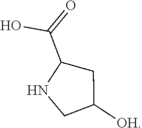

Hydroxyproline is produced in the body primarily from endogenous collagen turnover (Miyata et al., Proc Natl Acad Sci USA 111, 14406-14411, 2014). Using a unique metabolic tracer, ¹³C₅,¹⁵N-Hyp (all five carbons isotope and nitrogen atom labeled), it was determined that the level of Hyp turnover could be as high as 6-7 g/day (Riedel et al., Biochim Biophys Acta 1822, 1544-1552, 2012). Less than 5 mg of free Hyp is excreted in urine each day, indicating that most of the Hyp is metabolized (Belostotsky et al., J Mol Med (Berl) 90, 1497-1504, 2012). This significant metabolic load could contribute up to 25% of the endogenous oxalate produced (Phang et al., (2001) Disorders of proline and hydroxyproline metabolism. in The Metabolic and Molecular Bases of Inherited Disease (Scriver, C. R., Beaudet, A. L., Sly, W. S., Vallee, D., Childs, B., Kinzler, K. W., and Vogelstein, B. eds.), McGraw-Hill, New York. pp 1821-1838). The biological reason why Hyp metabolism occurs is not clear, although it does enable some pyruvate to feed back into other pathways.

Hyp is metabolized primarily in the mitochondria of the liver and renal cortical tissue (Kivirikko, Int Rev Connect Tissue Res 5, 93-163, 1970; Atlante et al., Biochem Biophys Res Commun 202, 58-64, 1994; Monico et al., Clin J Am Soc Nepthrol 6, 2289-2295, 2011; Wold et al., J Food Sc 64, 377-383, 1999). Diet can also be a source of collagen. For example, a quarter pound hamburger rich in gristle could contain as much as 6 grams of collagen, yielding 780 mg of Hyp (Khan et al., J Urol 184, 1189-1196, 2010). In fact, dietary Hyp can significantly increase oxalate production in humans and lead to hyperoxaluria in mouse and rat models (Khan et al., Kidney Int 70, 914-923, 2006; Valle et al., J Clin Invest 64, 1365-1370, 1979; Adams et al., Annu Rev Biochem 49, 1005-1061, 1980).

FIG. 1 presents the Hyp catabolic pathway and the metabolism of glyoxylate and glycolate. The Hyp pathway involves four enzymatic reactions (Miyata et al., Proc Natl Acad Sci USA 111, 14406-14411, 2014; Efron et al., New Engl J Med 272, 1299-1309, 1965; Pelkonen et al., New Engl J Med 283, 451-456, 1970). The first step of the pathway is the flavin FAD-dependent oxidation of Hyp to Δ¹-pyrroline-3-hydroxy-5-carboxylate (3-OH-P5C) by HYPDH. The 3-OH-P5C intermediate is converted to 4-hydroxy-glutamate (4-OH-Glu) by 1P5C dehydrogenase (1P5CDH), an NAD+-dependent enzyme shared with the proline degradation pathway (Efron et al., supra). Aspartate aminotransferase (AspAT) utilizes oxaloacetate to convert 4-OH-Glu to 4-hydroxy-2-oxoglutarate (HOG). HOG is then cleaved by the unique HOG aldolase (HOGA) into two fragments, glyoxylate and pyruvate. The glyoxylate can then be converted to glycolate and glycine via glyoxylate reductase (GR) and alanine:glyoxylate aminotransferase (AGT), respectively. Glycolate can be converted back into glyoxylate by glycolate oxidase (GO).

AGT, GR, and HOGA are mutated within primary hyperoxaluria patients (PH type 1, 2, and 3, respectively). For PH1 and PH2 patients, the glyoxylate produced from Hyp could exacerbate the already high levels of glyoxylate, and increase oxalate production via the lactate dehydrogenase (LDH). For PH3 patients, HOGA is inactivated, leading to a buildup of HOG (Riedel et al., Biochim Biophys Acta 1822, 1544-1552, 2012; Belostotsky et al., J Mol Med (Berl) 90, 1497-1504, 2012). Recent studies identified that HOG can inhibit GR, potentially leading to a PH2-like phenotype (Riedel et al., Biochim Biophys Acta 1822, 1544-1552, 2012). In contrast, hydroxyprolinemia, caused by deficiencies in HYPDH, is not associated with any overt consequences, and Hyp is safely excreted without being degraded (Curhan et al., Kidney Int 73, 489-496, 2008; Roy et al., Nature Protoc 5, 725-738, 2010). In addition, glycolic aciduria, caused by deficiencies in GO, is not associated with any overt consequences, and glycolate can be excreted (Frishberg et al. J Med Genet 51, 526-529, 2014).

Thus, and without wishing to be bound by theory, inhibition of GO and HYPDH enzymatic activities by a combination of small molecule inhibitors is not expected to lead to any adverse side effects, and will block the formation of glyoxylate and oxalate from glycolate and Hyp for all PH patient types and the buildup of HOG, 4-OH-Glu and dihydroxy-glutarate for PH3 patients. Inhibition of GO and HYPDH is also expected to help idiopathic stone formers and other individuals with high urinary oxalate levels, such as those that have undergone gastric bypass surgery. For the latter, there is a significant increase in stone formation that may benefit from prophylactic treatment post surgery. While the exact origins of the oxalate in these patients has not been determined, inhibition of GO and HYPDH will decrease glyoxylate and oxalate levels, which will ultimately reduce the glyoxylate and oxalate burden in them.

### 1. Active Compounds

Active compounds as described herein can be prepared in accordance with known procedures or variations thereof that will be apparent to those skilled in the art.

As will be appreciated by those of skill in the art, the active compounds of the various formulas disclosed herein may contain chiral centers, e.g., asymmetric carbon atoms, and the present disclosure is inclusive of both: (i) racemic mixtures of the active compounds, and (ii) enantiomeric forms of the active compounds. The resolution of racemates into enantiomeric forms can be done in accordance with known procedures in the art. For example, the racemate may be converted with an optically active reagent into a diastereomeric pair, and the diastereomeric pair subsequently separated into the enantiomeric forms.

Also included in active compounds disclosed herein are tautomers (e.g., tautomers of triazole, imidazole and/or pyrazole) and rotamers.

As described herein, certain groups or portions of the compounds of the invention may optionally be substituted with one or more substituents, such as those illustrated generally herein. In general, the term “substituted” refers to the replacement of hydrogen in a given structure with a substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formulation of stable compounds. “Stable” as used herein refers to a chemically feasible compound that is not substantially altered when kept at a temperature of 40° C. or less, in the absence of moisture or other chemically reactive conditions, for at least a week.

As used herein in the accompanying chemical structures, “H” refers to a hydrogen atom. “C” refers to a carbon atom. “N” refers to a nitrogen atom. “S” refers to a sulfur atom.

The term “hydroxy,” as used herein, refers to a group —OH.

“Carbonyl” is a group having a carbon atom double-bonded to an oxygen atom (C═O).

“Carboxy” as used herein refers to a group —COOH or —COO⁻.

“Amine” or “amino” refers to a group —NH₂.

“Halo” is a halogen group selected from the group consisting of fluoro (—F), choro (—Cl), bromo (—Br), and iodo (—I). “Haloalkyl” is a halogen group connected to the parent compound by an alkyl group.

“Alkyl,” as used herein, refers to a saturated straight or branched chain, or cyclic hydrocarbon containing from 1 to 10 carbon atoms. Representative examples of alkyl include, but are not limited to, methyl (Me), ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, cyclopropyl, cyclobutyl, cyclopentyl, cyclohexyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, cyclopropyl, cyclobutyl, and the like. The alkyl may be optionally substituted with one or more suitable substituents, such as halo, hydroxy, carboxy, amine, etc.

“Alkenyl,” as used herein, refers to a straight or branched chain hydrocarbon containing from 2 to 10 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 2 to 4 carbon atoms and at least one carbon-carbon double bond. The alkenyl may be optionally substituted with one or more suitable substituents, such as halo, hydroxy, carboxy, amine, etc.

“Alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like. “Lower alkynyl” as used herein, is a subset of alkynyl and refers to a straight or branched chain hydrocarbon group containing from 2 to 4 carbon atoms at least one carbon-carbon triple bond. The alkynyl may be optionally substituted with one or more suitable substituents, such as halo, hydroxy, carboxy, amine, etc.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, and the like. As noted, in some embodiments, the aryl has two aromatic rings, which rings are fused or directly adjoining. Examples include, but are not limited to, biphenyl, napthyl, azulenyl, etc. The aryl may be optionally substituted with one or more suitable substituents, such as alkyl, halo, hydroxy, carboxy, amine, etc.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, and the like. As noted, in some embodiments, the heteroaryl has two aromatic rings, which rings are fused or directly adjoining. Examples include, but are not limited to, benzothiophene, benzofuran, indole, benzoimidazole, benzthiazole, quinoline, isoquinoline, quinazoline, quinoxaline, phenyl-pyrrole, phenyl-thiophene, etc. The heteroaryl may be optionally substituted with one or more suitable substituents, such as alkyl, halo, hydroxy, carboxy, amine, etc.

A “pharmaceutically acceptable salt” is a salt that retains the biological effectiveness of the free acids or bases of a specified compound and that is not biologically or otherwise undesirable. Examples of pharmaceutically acceptable salts may include sulfates, pyrosulfates, bisulfates, sulfites, bisulfates, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

A “prodrug” is a compound that is converted under physiological conditions or by solvolysis or metabolically to a compound that is pharmaceutically active. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein in their entirety. See also Huttunen et al., “Prodrugs—from Serendipity to Rational Design,” Pharmacological Reviews 63(3):750-771 (2011), which is incorporated by reference herein. Example prodrugs include, but are not limited to, the addition of/conversion to phosphate(s), amino acid esters, amino acid amides, sugar derivatives, alkyl or aryl esters, etc., at an —OH, —SH, —NH or —COOH group of the parent active compound.

### A. HYPDH Inhibitors

Provided herein as active compounds according to some embodiments are HYPDH inhibitor compounds of Formula I:

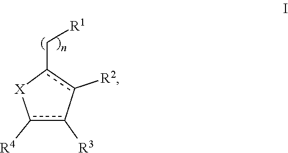

wherein:

X is O, S, NH, NMe or CR^{x}R^{y}, wherein R^{x} and R^{y} are each independently selected from H, alkyl or halo;

n is 0, 1, 2, 3, 4, 5 or 6;

R¹ is selected from the group consisting of: alkyl, alkenyl, alkynyl, aryl, halo, hydroxy, amine and carboxy;

R² is selected from the group consisting of: H, alkyl (e.g., lower alkyl), hydroxy, amine, and ═O; or R² is R^{2a}R^{2b}, wherein R^{2a} and R^{2b} are each independently selected from alkyl (e.g., lower alkyl) and hydroxy;

R³ is selected from the group consisting of: hydroxy, amine, and ═O; or R³ is R^{3a}R^{3b}, wherein R^{3a} and R^{3b} are each independently hydroxy; and

R⁴ is selected from the group consisting of: H, alkyl (e.g., lower alkyl), and hydroxy; or R⁴ is R^{4a}R^{4b} wherein R^{4a} and R^{4b} are each independently selected from alkyl (e.g., lower alkyl), hydroxy, and halo, wherein said alkyl may be unsubstituted or substituted 1, 2 or 3 times with hydroxy,

or a pharmaceutically acceptable salt or prodrug thereof.

In some embodiments of Formula I, X is O, or CR^{x}R^{y}.

In some embodiments of Formula I, n is 0 and/or R¹ is carboxy.

In some embodiments of Formula I, R² and/or R⁴ is selected from the group consisting of: H and lower alkyl.

In some embodiments of Formula I, R³ is hydroxy.

In some embodiments, of Formula I, the compound is a compound of Formula I(A):

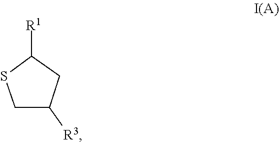

wherein:

R¹ is selected from the group consisting of: alkyl, alkenyl, alkynyl, aryl, halo, hydroxy, amine and carboxy; and

R³ is selected from the group consisting of: H, hydroxy, amine, and ═O; or R³ is R^{3a}R^{3b}, wherein R^{3a} and R^{3b} are each independently selected from alkyl (e.g., lower alkyl) and hydroxy;

or a pharmaceutically acceptable salt or prodrug thereof.

In some embodiments of Formula I(A), R¹ is carboxy and/or R³ is hydroxy or R^{3a}R^{3b}, wherein R^{3a} and R^{3b} are each hydroxy.

Also provided herein are HYPDH inhibitor compounds of Formula II:

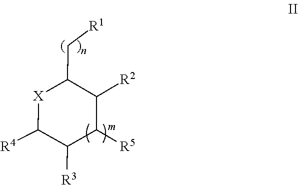

wherein:

X is O, NH, NMe or CR^{x}R^{y}, wherein R^{x} and R^{y} are each independently selected from H, alkyl or halo;

n is 0, 1, 2, 3, 4, 5 or 6;

m is 0, 1, 2, or 3;

R¹ is selected from the group consisting of: alkyl, alkenyl, alkynyl, aryl, halo, hydroxy, amine and carboxy;

R² is selected from the group consisting of: H, alkyl (e.g., lower alkyl), hydroxy, amine, and ═O; or R² is R^{2a}R^{2b}, wherein R^{2a} and R^{2b} are each independently selected from alkyl (e.g., lower alkyl) and hydroxy;

R³ is selected from the group consisting of: H, hydroxy, amine, and ═O; or R³ is R^{3a}R^{3b}, wherein R^{3a} and R^{3b} are each independently selected from alkyl (e.g., lower alkyl) and hydroxy;

R⁴ is selected from the group consisting of: H, alkyl (e.g., lower alkyl), and hydroxy; or R⁴ is R^{4a}R^{4b} wherein R^{4a} and R^{4b} are each independently selected from alkyl (e.g., lower alkyl), hydroxy, and halo, wherein said alkyl may be unsubstituted or substituted 1, 2 or 3 times with hydroxy; and

each R⁵ is independently selected from the group consisting of: H, alkyl (e.g., lower alkyl), hydroxy, amine, and ═O; or R⁵ is R^{5a}R^{5b} wherein R^{5a} and R^{5b} are each independently selected from alkyl (e.g., lower alkyl) and hydroxy; or R² and an adjacent R⁵ are taken together to form an aryl or heteroaryl,

or a pharmaceutically acceptable salt or prodrug thereof.

In some embodiments of Formula II, X is O, NH, NMe or CR^{x}R^{y}.

In some embodiments of Formula II, n is 0 and/or R¹ is hydroxy.

In some embodiments of Formula II, R² and/or R⁴ is selected from the group consisting of: H and lower alkyl.

In some embodiments of Formula II, R³ is hydroxy.

In some embodiments of Formula II, R² is selected from the group consisting of: H, hydroxy, and lower alkyl.

Further provided herein are HYPDH inhibitor compounds of Formula III:

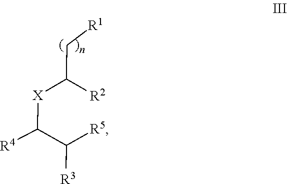

wherein:

X is O, S, NH, NMe or CR^{x}R^{y}, wherein R^{x} and R^{y} are each independently selected from H, alkyl or halo;

n is 0, 1, 2, 3, 4, 5 or 6;

R¹ is selected from the group consisting of: alkyl, alkenyl, alkynyl, aryl, halo, hydroxy, amine and carboxy;

R² is selected from the group consisting of: H, alkyl (e.g., lower alkyl), hydroxy, amine, and ═O; or R² is R^{2a}R^{2b}, wherein R^{2a} and R^{2b} are each independently selected from alkyl (e.g., lower alkyl) and hydroxy;

R³ is selected from the group consisting of: H, hydroxy, amine, and ═O; or R³ is R^{3a}R^{3b}, wherein R^{3a} and R^{3b} are each independently selected from alkyl (e.g., lower alkyl) and hydroxy;

R⁴ is selected from the group consisting of: H, alkyl (e.g., lower alkyl), and hydroxy; or R⁴ is R^{4a}R^{4b} wherein R^{4a} and R^{4b} are each independently selected from alkyl (e.g., lower alkyl), hydroxy, and halo, wherein said alkyl may be unsubstituted or substituted 1, 2 or 3 times with hydroxy; and

R⁵ is independently selected from the group consisting of: H, alkyl (e.g., lower alkyl), hydroxy, amine, and ═O; or R⁵ is R^{5a}R^{5b} wherein R^{5a} and R^{5b} are each independently selected from alkyl (e.g., lower alkyl) and hydroxy,

or a pharmaceutically acceptable salt or prodrug thereof.

In some embodiments of Formula III, X is NH.

In some embodiments of Formula III, n is 0 and/or R¹ is hydroxy.

In some embodiments of Formula III, R² and/or R⁴ is selected from the group consisting of: H and lower alkyl.

In some embodiments of Formula III, R³ is hydroxy.

### B. GO Inhibitors

Provided herein as active compounds according to some embodiments are GO inhibitor compounds of Formula IV:

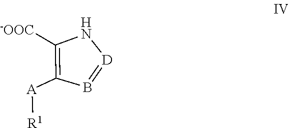

wherein:

A is CH₂ or S;

B is CH or N;

D is CH or N; and

R¹ is aryl or heteroaryl, wherein said aryl or heteroaryl has two aromatic rings, which rings are fused or directly adjoining,

or a pharmaceutically acceptable salt or prodrug thereof.

In some embodiments of Formula IV, A is CH₂. In some embodiments, A is S. In some embodiments, B is CH. In some embodiments, B is N. In some embodiments, D is CH. In some embodiments, D is N.

In some embodiments of Formula IV, R¹ is benzothiophene or biphenyl.

In some embodiments of Formula IV, R¹ is selected from the group consisting of:

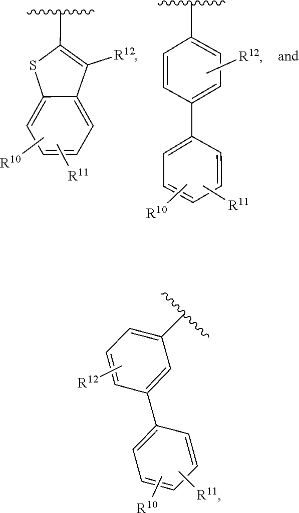

wherein R¹⁰, R¹¹ and R¹² are each independently selected from the group consisting of: H, alkyl, halo and haloalkyl.

Also provided are GO inhibitor compounds of Formula V:

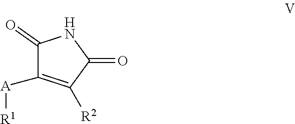

wherein:

A is CH₂ or S;

R¹ is aryl or heteroaryl, wherein said aryl or heteroaryl has two aromatic rings, which rings are fused or directly adjoining; and

R² is H or OH,

or a pharmaceutically acceptable salt or prodrug thereof.

In some embodiments of Formula V, A is CH₂. In some embodiments, A is S.

In some embodiments of Formula V, R¹ is benzothiophene or biphenyl.

In some embodiments of Formula V, R¹ is selected from the group consisting of:

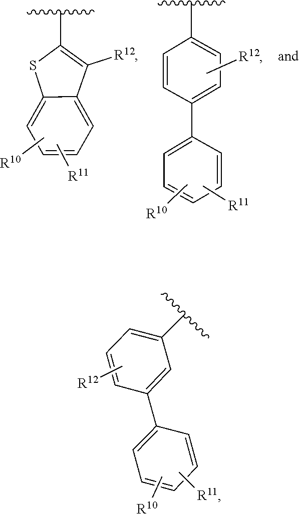

wherein R¹⁰, R¹¹ and R¹² are each independently selected from the group consisting of: H, alkyl, halo and haloalkyl.

GO inhibitors also include those provided in U.S. Pat. No. 4,178,386 to Williams et al.; U.S. Pat. Nos. 4,428,956, 4,431,652 and 4,537,902 to Cragoe, Jr. et al.

### C. Other Active Agents for the Treatment of Kidney Stones

Combination treatments as taught herein may include another active agent(s) for treatment of kidney stones. Treatments may include a cysteine precursor inhibitor of hepatic oxalate synthesis such as (L)-oxothiazolidine-4-carboxylate (OTZ). See, e.g., “Primary hyperoxaluria type 1,” Kidney International 55:2533-2547, 1999. Treatments may include a calcium oxalate crystallization inhibitor, such as sodium or potassium citrate, sodium or potassium bicarbonate, phosphate such as orthophosphate, etc. Treatments may include an AGT cofactor such as pyridoxine (e.g., pyridoxal-5-phosphate). Treatments may include a kidney sodium glucose transporter inhibitor, carbohydrate, and/or ADH antagonist such as that of U.S. Pat. Nos. 6,414,126 and 6,515,117 to Ellsworth et al. (e.g., dapagliflozin); U.S. Pat. No. 6,774,112 to Gougoutas; U.S. Pat. No. 8,603,989 to Halperin, GSK189075 (remogliflozin), GW869682, etc. Treatments may include calcium channel blockers to decrease the amount of calcium in the urine (e.g., nifedipine). Treatments may include alpha-1 blockers to promote stone passage (e.g., tamulosin).

Treatment may include a diuretic such as a thiazide diuretic. Diuretics may include, for example, ammonium chloride, glycerin, isosorbide, dichlorphenamide, methazolamide, acetazolamide, acetazolamide sodium, benzothiadiazine, bendroflumethiazide, benzthiazide, chlorthalidone, chlorothiazide, cyclothiazide, hydrochlorothiazide, hydroflumethiazide, indapamide, methylclothiazide, metolazone, polythiazide, quinethazone, tricholomethiazide, amiloride hydrochloride, spironolactone, triamterene, bumetamide, ethacrynic acid, ethacrynate sodium, furosemide, and torsemide (Remington: the Science and Practice of Pharmacy, 21st ed. 2005, Lippincott Williams & Wilkins, Philadelphia, Pa.).

### 2. Formulations

The active compounds described herein may be formulated for administration in a pharmaceutical carrier in accordance with known techniques. See, e.g., Remington, The Science and Practice of Pharmacy (9^{th} Ed. 1995). In the manufacture of a pharmaceutical formulation according to the invention, the active compound (including the physiologically acceptable salts or prodrugs thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active agent. One or more active agents may be incorporated in the formulations of the invention, which may be prepared by any of the well-known techniques of pharmacy comprising admixing the components, optionally including one or more accessory ingredients.

The pharmaceutical compositions may also contain other additives, such as pH-adjusting additives. In particular, useful pH-adjusting agents include acids, such as hydrochloric acid, bases and/or buffers, such as sodium lactate, sodium acetate, sodium phosphate, sodium citrate, sodium borate, or sodium gluconate. Further, the compositions may contain preservatives. Useful preservatives include methylparaben, propylparaben, benzoic acid and benzyl alcohol.

The formulations may comprise nanoparticles, such as biodegradable polymers and/or liposome-forming material, for encapsulation and/or delivery of the active agent(s). See, e.g., WO 2014/201312 to Wang et al.; Cho and Jung, “Supramolecular Complexation of Carbohydrates for the Bioavailability Enhancement of Poorly Soluble Drugs,” Molecules 20:19620-19646, 2015; Nogueira et al., “Design of liposomal formulations for cell targeting,” Colloids Surf B Biointerfaces 136:514-526, 2015. In some embodiments, liver-targeting nanoparticles may be used for specific delivery of active agent(s) acting at the liver. See, e.g., U.S. 2015/0150994 to Hahn et al.; U.S. 2008/0138394 to Kim et al. In some embodiments, kidney-targeting nanoparticles may be used for specific delivery of active agent(s) acting at the kidney. See, e.g., U.S. Pat. No. 8,318,199 to Kim et al.; U.S. 2012/0196807 to Nakamura et al.

In some embodiments, the active agent(s) may be provided in a controlled-release or sustained-release formulation. See, e.g., Grinyo and Petruzzelli, “Once-daily LCP-Tacro MeltDose tacrolimus for the prophylaxis of organ rejection in kidney and liver transplantations,” Expert Review of Clinical Immunology 10(12):1567-1579, 2014 (Erratum: Expert Review of Clinical Immunology 11(4):547, 2015).

The two or more active compounds may be provided in the same formulation or composition, or in different formulations or compositions. Two or more formulations or compositions may also be provided as a kit comprising the same. For example, a kit may comprise a composition or formulation comprising a HYPDH inhibitor, a composition or formulation comprising a GO inhibitor, and optionally a composition or formulation comprising another agent for the treatment of kidney stones.

Formulations of the invention may include those suitable for oral, buccal (sub-lingual), parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound being used.

Formulations suitable for oral administration may be presented in discrete units, such as capsules, cachets, lozenges, or tablets, each containing a predetermined amount of the active compound(s); as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for oral administration also include food product formulations, such as a nutritional bar or an animal feed (e.g., pet food such as dog or cat food). Food product formulations may include one or more of carbohydrates such as wheat, corn rice, barley or oats, dairy products such as milk, oils such as canola oil or soybean oil, flavorants such as sugar or syrup, coloring, chocolate, preservatives, etc. Pet food formulations, in particular, may include meat, poultry, fish or other animal-derived components such as eggs.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s) in a unit dosage form in a sealed container. The active compound(s) may be provided in the form of a lyophilizate which is capable of being reconstituted with a suitable pharmaceutically acceptable carrier to form a liquid composition suitable for injection thereof into a subject.

When the active compound(s) is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

The unit dosage form typically comprises from about 1 mg, 5 mg, 10 mg, 100 mg, 250 mg, 500 mg, 1 gram, 5 grams, 10 grams, or any ranges therein, of the active compound(s), depending on the subject being treated (e.g., human or non-human mammalian subject). In some embodiments, the unit dosage form is in the range of 500 mg to 10 grams, keeping in mind that a good portion of the active compound(s) may not be absorbed upon administration (e.g., oral administration).

### 3. Combination Treatments

As used herein, the administration of two or more active agents “in combination” means that the two are administered closely enough in time that the administration of or presence of one alters, adds to and/or enhances the biological effects of the other. The therapies may be administered simultaneously (concurrently) or sequentially.

Simultaneous administration of the agents may be carried out by mixing the agents prior to administration, including the agents in the same formulation or food product, or by administering the agents at the same point in time but at different anatomic sites or using different routes of administration, or administered at times sufficiently close that the results observed are indistinguishable from those achieved when the agents are administered at the same point in time.

Sequential administration of the agents may be carried out by administering the agents at different points in time, e.g., an active agent at some point in time prior to or after administration of one or more other active agents, such that the administration of agent enhances the therapeutic effect of the treatment. In some embodiments, an active agent is administered at some point in time prior to the initial administration of another active agent. Alternatively, the other active agent may be administered at some point in time prior to the administration of the active agent, and optionally, administered again at some point in time after the administration of an active agent.

The present invention is explained in greater detail in the following non-limiting examples.

### EXAMPLES

### Example 1

Measurement of Hydroxyproline metabolism. Patients with PH1, PH2, and PH3 and normal subjects were placed on a 3-day controlled diet and infused in the fasted state with ¹⁵N-¹³C₅-Hyp at a constant rate (750 nmol/kg/h) for 6 h. Urine and plasma samples were collected hourly for analysis: total ¹³C-labelled Hyp and glycine by GC/MS; oxalate and glycolate by IC and IC/MS. The tracer has proven to work effectively and safely. The tracer did not change the pre-infusion and post-infusion total urinary oxalate excretion (e.g., 13±3 versus 9±4 mg/g creat/h for normal; 60±50 versus 40±29 mg/g creat/h for PH1; similar values for PH2 and PH3 samples).

A preliminary comparison of the enrichment of the tracer in plasma Hyp, urine oxalate, and urine glycolate reveals intriguing patterns and highlights the degree to which Hyp metabolism in PH1-3 patients is different. The preliminary data indicate that the plasma levels of ¹⁵N-¹³C₅-Hyp in PH1 and PH3 patients is enriched over controls (˜2-fold). This may suggest that Hyp turnover is slower in these patients; however, the range of values for the patients tested is quite wide, and overlaps with the control values (hence, the need to know which PH1 mutations are present and the treatment regimen). There is also the possibility that collagen breakdown by collagenases may be yielding a spectrum of peptides that may be metabolized more quickly and partition differently in plasma than free Hyp.

Notably, the proportion of the label in urine oxalate is significantly increased in all PH patient groups (2- to 8-fold), with PH3 being the highest. This observation supports that HOG is being broken down in PH3 patients by another pathway to glyoxylate/oxalate.

Altogether, these observations suggest that Hyp contributes up to 25% of urinary oxalate.

An increase in the level of urine oxalate, on the order of 3-5 mg/day, can have up to a 2-fold increase in stone disease risk (Zhang, BMC Bioinformatics 9, 40, 2008). Therefore, blocking HYPDH activity has the potential to decrease the amount of glyoxylate and oxalate produced endogenously by all three types of PH patients, and to markedly reduce their risk for stone formation and disease. Similarly, HYPDH inhibition may also benefit idiopathic stone formers and other individuals with high urinary oxalate levels, such as those that have undergone gastric bypass surgery. For the latter, there is a significant increase in stone formation that may benefit from prophylactic treatment post surgery. While the exact origins of the oxalate in these patients has not been determined, inhibition of HYPDH will decrease glyoxylate and oxalate levels, which will ultimately reduce the glyoxylate and oxalate burden in them.

### Example 2

Development of recombinant, human HYPDH and activity assay. Despite the identification of the Hyp pathway in rat and bovine kidneys and livers over 50 years ago, very little is known about human HYPDH (also known as PRODH2 and hydroxyproline oxidase, HPOX, in the literature) (Miyata et al., Proc Natl Acad Sci USA 111, 14406-14411, 2014; Efron et al., New Engl J Med 272, 1299-1309; Pelkonen et al., New Engl J Med 283, 451-456, 1970). In an effort to biochemically and structurally characterize human HYPDH, we have evaluated numerous expression constructs (>15) in Escherichia coli with N- and C-terminal truncations. These constructs exhibit different levels of protein production, solubility (i.e., inclusion body formation), FAD cofactor loading, and enzymatic activity. Only the constructs containing the residues 147-515 and 156-515 were >96% loaded with FAD+ and active.

Recombinant HYPDH: (1) displays typical FAD spectra upon oxidation and reduction, (2) exhibits kinetic parameters for the turnover of Hyp consistent with homologs (Zhang, BMC Bioinformatics 9, 40, 2008; Moxley et al., Biochemistry 51, 511-520, 2012; Moxley et al., Arch Biochem Biophys 516, 113-120, 2011; Srivastava et al., Proc Natl Acad Sci USA 107, 2878-2883, 2010), (3) is selective for Hyp and not Pro, (4) readily uses a variety of CoQ10 analogs as an electron acceptor during catalysis, and (5) binds Hyp with a K_{D} value of 125 μM, using an anaerobic titration of the FAD spectrum. These data represent the first biochemical data available for human HYPDH by any laboratory.

### Example 3

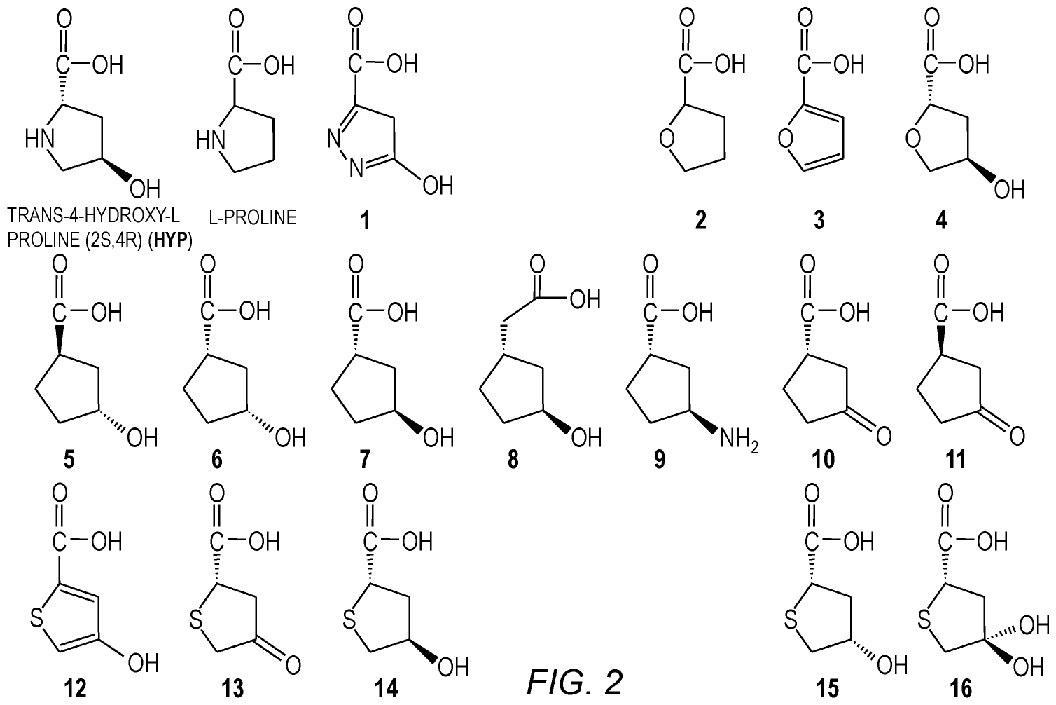

Identification and testing of inhibitors of HYPDH. Tested compounds are shown in FIG. 2. Some compounds were commercially available, and non-commercial compounds were synthesized on a fee-for-service basis contract with Jasco Pharmaceuticals (Woburn, Mass.). Compound 3 is not yet tested, and compound 4 is not yet synthesized.

Each inhibitor was pre-incubated with HYPDH for 5 min, and the reaction started by the addition of 600 mM Hyp. A range of concentrations was tested in order to determine the IC₅₀ value. Table 1 lists the potency of the compounds.

TABLE 1

| Cmpd | IC₅₀ (mM) |
|---|---|
| 1 | 2.9 ± 0.1 |
| 5 | 2.1 ± 0.1 |
| 2 | 1.5 ± 0.1 |
| 3 | ND^{a} |
| 4 | ND^{b} |
| 9 | 0.63 ± 0.01 |
| 6 | >10 |
| 7 | 0.60 ± 0.01 |
| 10 | 0.38 ± 0.01 |
| 11 | 0.37 ± 0.01 |
| 8 | 0.48 ± 0.01 |
| 13 | 0.32 ± 0.01 |
| 12 | <1 |
| 15 | 0.37 ± 0.01 |
| 14 | 0.33 ± 0.01 |
| 16 | 0.29 ± 0.01 |

^{a}Not soluble in buffer
^{b}Synthesis planned

Inhibitors of HYPDH were identified as compounds in which the nitrogen atom of the Hyp ring is changed to oxygen, carbon or sulfur. This substitution prevents ring oxidation and cleavage by HYPDH. The data indicate that the most potent compounds belong to the reduced thiophene class, closely followed by the cyclopentane analogs.

### Example 4

Further testing of inhibitors of HYPDH. Additional compounds are obtained, and tested in the same manner as in Example 3 above. These additional compounds may include:

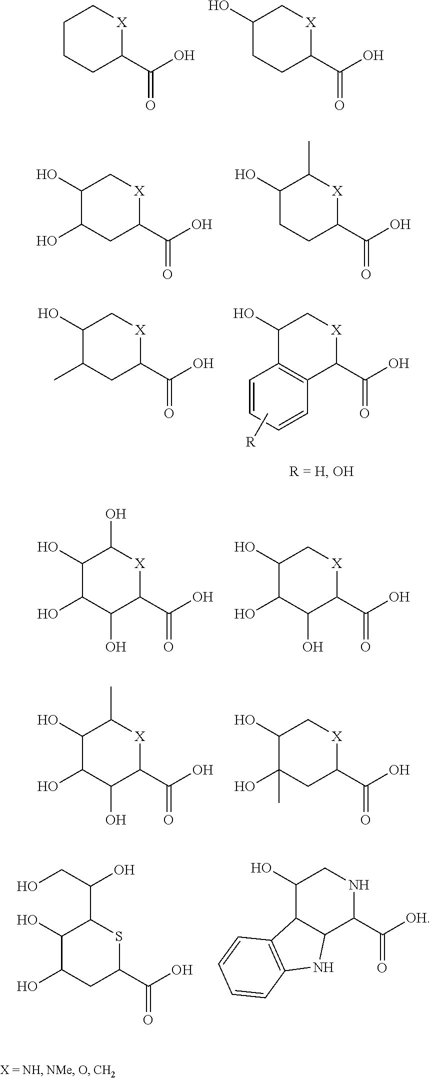

For example, additional compounds may include:

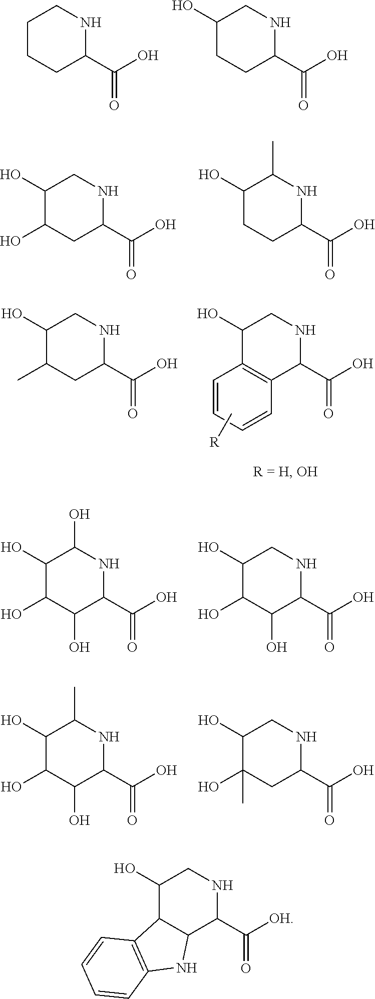

### Example 5

Glycolate oxidase (GO) inhibitor design. Based on crystal structures of human GO1 with CCPST and CDST as well as other biochemical data, GO inhibitors are designed to exploit one or more of the following interactions:

(1) force W110 to “flip” out of the active site, causing loop 4 to become disordered;

(2) protonated nitrogen at position 3 directly interacts with the catalytic residue His260;

(3) carboxylate interaction with one or both of two conserved Arg residues.

### Example 6

Example GO inhibitors. With the above considerations in mind, the following compounds are designed as GO inhibitors.

[chemical structure]

[chemical structure]

[chemical structure]

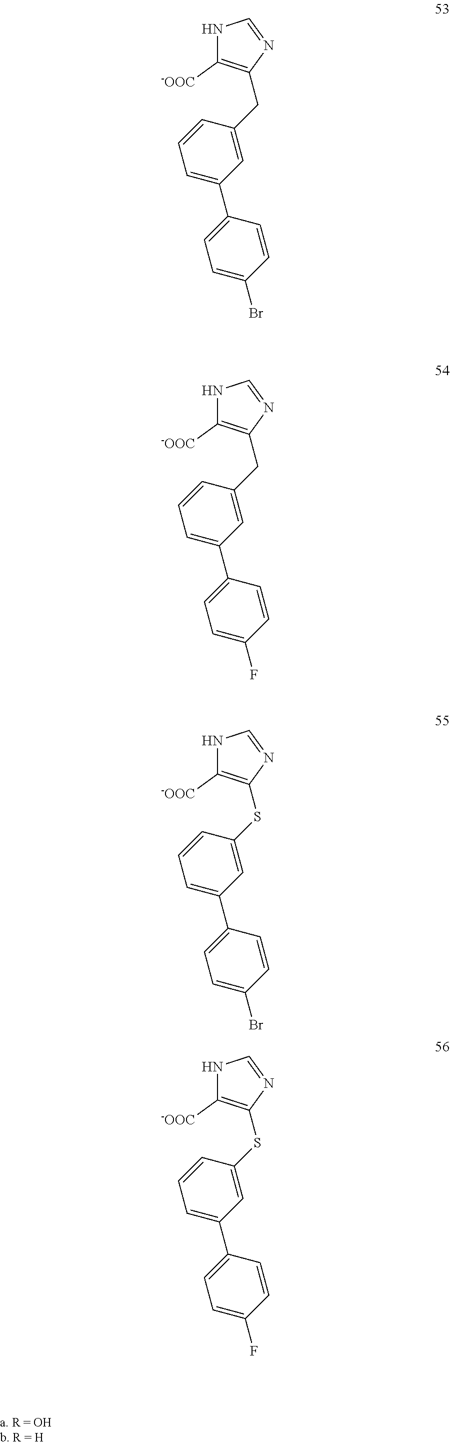

### Example 7

Testing of GO inhibitors. The inhibition of recombinant, human liver GO (the HAO1 gene product) is readily determined by a coupled assay that contains 2,6-dichloroindophenol (DCIP) (Murray et al. Biochemistry 47, 2439-2449, 2008). Briefly, GO is pre-incubated at 37° C. with or without inhibitor in 100 mM potassium phosphate pH 7.5 (0.1% DMSO final) for 5 mM. An aliquot of pre-warmed DCIP and glycolate is added to start the reaction (final concentration 75 μM DCIP, 3 mM glycolate). The reaction rate is determined by monitoring the decrease at 600 nm (extinction coefficient of 21 mM⁻¹ cm⁻¹). CDST inhibits GO with an apparent Ki of ˜15 nM.

### Example 8

Combination therapy with HYPDH inhibitor and GO inhibitor. Subjects are administered an HYPDH inhibitor in combination with a GO inhibitor to treat kidney stones. Subjects may be monitored by measurement of urinary oxalate excretion.

### LITERATURE CITED

Adams, E., and Frank, L. (1980) Metabolism of proline and the hydroxyprolines. Annu Rev Biochem 49, 1005-1061
Belostotsky, R., Pitt, J. J., and Frishberg, Y. (2012) Primary hyperoxaluria type III—a model for studying perturbations in glyoxylate metabolism. J Mol Med (Berl) 90, 1497-1504
Atlante, A., Passarella, S., and Quagliariello, E. (1994) Spectroscopic study of hydroxyproline transport in rat kidney mitochondria. Biochem Biophys Res Commun 202, 58-64
Curhan, G. C., and Taylor, E. N. (2008) 24-h uric acid excretion and the risk of kidney stones. Kidney Int 73, 489-496
Efron, M. L., Bixby, E. M., and Pryles, C. V. (1965) Hydroxyprolinemia. Ii. A Rare Metabolic Disease Due to a Deficiency of the Enzyme “Hydroxyproline Oxidase”. New Engl J Med 272, 1299-1309
Frishberg, Y., Zeharia, A., Lyakhovetsky, R. Bargal, R. and Belostotsky, R. (2014) Mutations in Hao1 encoding glycolate oxidase cause isolated glycoli aciduria. J Med Genet 51, 526-529
Murray, M. S., Holmes, R. P. and Lowther, W. T. (2008) Active site loop 4 movements with human glycoalte oxidase: implications for substrate specificity and drug design. Biochemistry 47, 2439-2449.
Jiang, J., Johnson, L. C., Knight, J., Callahan, M. F., Riedel, T. J., Holmes, R. P., and Lowther, W. T. (2012) Metabolism of [13C5]hydroxyproline in vitro and in vivo: implications for primary hyperoxaluria. Am J Physiol Gastrointest Liver Physiol 302, G637-643
Jones, J. M, Morrell, J. C. and Gould, S. J. (2000) Indentification and characterization of HAOX1, HOAX2, and HOAX3, three human peroxisome 2-hydroxy acid oxidases. J Biol Chem 275, 12590-12597
Khan, S. R., and Glenton, P. A. (2010) Experimental induction of calcium oxalate nephrolithiasis in mice. J Urol 184, 1189-1196
Khan, S. R., Glenton, P. A., and Byer, K. J. (2006) Modeling of hyperoxaluric calcium oxalate nephrolithiasis: experimental induction of hyperoxaluria by hydroxy-L-proline. Kidney Int 70, 914-923
Kivirikko, K. I. (1970) Urinary excretion of hydroxyproline in health and disease. Int Rev Connect Tissue Res 5, 93-163
Knight, J., and Holmes, R. P. (2005) Mitochondrial hydroxyproline metabolism: implications for primary hyperoxaluria. Am J Nephrol 25, 171-175
Knight, J., Holmes, R. P., Cramer, S. D., Takayama, T., and Salido, E. (2012) Hydroxyproline metabolism in mouse models of primary hyperoxaluria. Am J Physiol-Renal 302, F688-693
Knight, J., Jiang, J., Assimos, D. G., and Holmes, R. P. (2006) Hydroxyproline ingestion and urinary oxalate and glycolate excretion. Kidney Int 70, 1929-1934
Lipinski, C. A., Lombardo, F., Dominy, B. W., and Feeney, P. J. (2001) Experimental and computational approaches to estimate solubility and permeability in drug discovery and development settings. Adv Drug Deliv Rev 46, 3-26
Miyata, N., Steffen, J., Johnson, M. E., Fargue, S., Danpure, C. J., and Koehler, C. M. (2014) Pharmacologic rescue of an enzyme-trafficking defect in primary hyperoxaluria 1. Proc Natl Acad Sci USA 111, 14406-14411
Monico, C. G., Rossetti, S., Belostotsky, R., Cogal, A. G., Herges, R. M., Seide, B. M., Olson, J. B., Bergstrahl, E. J., Williams, H. J., Haley, W. E., Frishberg, Y., and Milliner, D. S. (2011) Primary hyperoxaluria type III gene HOGA1 (formerly DHDPSL) as a possible risk factor for idiopathic calcium oxalate urolithiasis. Clin J Am Soc Nepthrol 6, 2289-2295
Moxley, M. A., and Becker, D. F. (2012) Rapid reaction kinetics of proline dehydrogenase in the multifunctional proline utilization A protein. Biochemistry 51, 511-520
Moxley, M. A., Tanner, J. J., and Becker, D. F. (2011) Steady-state kinetic mechanism of the proline:ubiquinone oxidoreductase activity of proline utilization A (PutA) from Escherichia coli. Arch Biochem Biophys 516, 113-120
Ostrander, E. L., Larson, J. D., Schuermann, J. P., and Tanner, J. J. (2009) A Conserved Active Site Tyrosine Residue of Proline Dehydrogenase Helps Enforce the Preference for Proline over Hydroxyproline as the Substrate. Biochemistry 48, 951-959
Pelkonen, R., and Kivirikko, K. I. (1970) Hydroxyprolinemia: an apparently harmless familial metabolic disorder. New Engl J Med 283, 451-456
Pemberton, T. A., and Tanner, J. J. (2013) Structural basis of substrate selectivity of Delta(1)-pyrroline-5-carboxylate dehydrogenase (ALDH4A1): semialdehyde chain length. Arch Biochem Biophys 538, 34-40
Phang, J. M., Hu, C. A., and Valle, D. (2001) Disorders of proline and hydroxyproline metabolism in The Metabolic and Molecular Bases of Inherited Disease (Scriver, C. R., Beaudet, A. L., Sly, W. S., Vallee, D., Childs, B., Kinzler, K. W., and Vogelstein, B. eds.), McGraw-Hill, New York. pp 1821-1838
Riedel, T. J., Johnson, L. C., Knight, J., Hantgan, R. R., Holmes, R. P., and Lowther, W. T. (2011) Structural and Biochemical Studies of Human 4-hydroxy-2-oxoglutarate Aldolase: Implications for Hydroxyproline Metabolism in Primary Hyperoxaluria. PLoS One 6, e26021
Riedel, T. J., Knight, J., Murray, M. S., Milliner, D. S., Holmes, R. P., and Lowther, W. T. (2012) 4-Hydroxy-2-oxoglutarate aldolase inactivity in primary hyperoxaluria type 3 and glyoxylate reductase inhibition. Biochim Biophys Acta 1822, 1544-1552
Roy, A., Kucukural, A., and Zhang, Y. (2010) I-TASSER: a unified platform for automated protein structure and function prediction. Nature Protoc 5, 725-738
Srivastava, D., Schuermann, J. P., White, T. A., Krishnan, N., Sanyal, N., Hura, G. L., Tan, A., Henzl, M. T., Becker, D. F., and Tanner, J. J. (2010) Crystal structure of the bifunctional proline utilization A flavoenzyme from Bradyrhizobium japonicum. Proceedings of the National Academy of Sciences of the United States of America 107, 2878-2883
Tallarita, E., Pollegioni, L., Servi, S., and Molla, G. (2012) Expression in Escherichia coli of the catalytic domain of human proline oxidase. Protein Expres Purif 82, 345-351
Valle, D., Goodman, S. I., Harris, S. C., and Phang, J. M. (1979) Genetic evidence for a common enzyme catalyzing the second step in the degradation of proline and hydroxyproline. J Clin Invest 64, 1365-1370
White, T. A., Krishnan, N., Becker, D. F., and Tanner, J. J. (2007) Structure and kinetics of monofunctional proline dehydrogenase from Thermus thermophilus. J Biol Chem 282, 14316-14327
Williams, I., and Frank, L. (1975) Improved chemical synthesis and enzymatic assay of delta-1-pyrroline-5-carboxylic acid. Anal Biochem 64, 85-97
Williams, H. J., Williams, N., Spurlock, G., Norton, N., Zammit, S., Kirov, G., Owen, M. J., and O'Donovan, M. C. (2003) Detailed analysis of PRODH and PsPRODH reveals no association with schizophrenia. Am J Med Genet B Neuropsychiatr Genet 120B, 42-46
Willis, A., Bender, H. U., Steel, G., and Valle, D. (2008) PRODH variants and risk for schizophrenia Amino Acids 35, 673-679
Wold, J. P., Lundby, F., and Egelandsdel, B. (1999) Quantification of connective tissue (hydroxyproline) in ground beef by autofluorescence spectroscopy. J Food Sc 64, 377-383
Zhang, Y. (2008) I-TASSER server for protein 3D structure prediction. BMC Bioinformatics 9, 40

### Example 9

Mouse models. Mice that do not express HYPDH, and mice that do not express GO, have been generated.

The Prodh2 (HYPDH) deficient animals developed normally and exhibited similar behavior to wild-type litter mates. The genotype of each mouse was confirmed by PCR analysis from a tail snip. Liver, kidney and isolated liver mitochondria were analyzed by western analysis. These tests confirmed that the Prodh2 homozygous mouse did not contain HYPDH in any of the samples. As expected, HYPDH is expressed in the liver and kidney of Wt and heterozygous (Htz) mice.

Mice lacking HYPDH appear normal apart from an increased urinary Hyp excretion and elevated plasma Hyp level. Male mice lacking HYPDH, and fed an oxalate-free diet, excrete ˜20% less urinary oxalate than wild type litter mates; however, female mice deficient in HYPDH excrete similar levels of oxalate compared to Wt litter mates. This data supports that Hyp can contribute significantly to urinary oxalate in mice.

Extreme dietary Hyp has been used by several groups to produce hyperoxaluria in mice, rats and pigs. The urinary oxalate levels of Prodh2 knockout mice challenged with 1% Hyp was impacted minimally. These data further support that HYPDH is a suitable target for oxalate reduction therapy.

The Hao1 (GO) deficient animals developed normally and exhibited similar behavior to wild-type litter mates. The genotype of each mouse was confirmed by PCR analysis from a tail snip. Liver was analyzed by western analysis. These tests confirmed that the Hao1 homozygous mouse did not contain GO in any of the samples. As expected, GO is not present in the kidney of all mouse strains.

Mice lacking GO appear normal apart from an increased urinary glycolate excretion and elevated plasma glycolate level. Male mice lacking GO excreted ˜1.4 fold more urinary oxalate than wild type litter mates; however, female Hao1 deficient mice show no significant difference in urinary oxalate excretion compared to Wt litter mates. It is noted that this finding with male Hao1 deficient mice is not consistent with data recently published by Dr. Salido's group that showed no difference in urinary oxalate excretion between Wt and Hao1 deficient male mice. However, the diet used by Dr. Salido's group was different from that used in this study.

Heterozygous Prodh2 and Hao1 mouse strains showed reduced expression of protein as measured by western Blot. While urinary oxalate excretion in the Htz mice compared to their respective wild-type litter mates was not statistically significant, the Prodh2 Htz mice excreted on average 7% less urinary oxalate than Wt litter mates. This data supports HYPDH expression levels can modulate urinary oxalate levels.

Given the cycle of glycolate-glyoxylate interconversion that will occur via GO and glyoxylate reductase activities in hepatocytes lacking alanine-glyoxylate aminotransferase (AGT), inhibition of GO is likely to reduce oxalate synthesis in PH1 patients. The contribution of glycolate to oxalate synthesis in humans with functional AGT activity is not known; however, individuals lacking GO appear normal. Frishberg et al., J Med Genet 51(8):526-9 (2014). Therefore, strategies to reduce GO activity in combination with HYPDH inhibition may provide the most benefit for reducing urinary oxalate excretion in patients with calcium oxalate kidney stone disease. The increase in plasma glycolate that occurs with GO inhibition may also be muted significantly with HYPDH inhibition, considering Hyp contributes to ˜60% of urinary glycolate excretion in normal individuals. Thus, the combination of less synthesis of glycolate (inhibition of HYPDH) and inhibiting GO activity, which converts glycolate back to glyoxylate, may be a more powerful approach to limit oxalate synthesis in patients with calcium oxalate kidney stone disease.

### Example 10

Hyp infusion studies. Consistent with Prodh2 homozygote mice excreting 20% less urinary oxalate, intravenous infusion studies with ¹⁵N-¹³C₅-hydroxyproline in healthy human subjects indicate that Hyp contributes ˜20% to urinary oxalate and ˜60% to urinary glycolate excretion. These data established that Hyp metabolism could account for up to 80% of endogenously-produced glyoxylate. Since glyoxylate is the precursor to oxalate, blocking HYPDH activity should have a significant impact on urinary oxalate. The metabolic-tracer infusion studies in PH2 and PH3 patients illustrate that defects in glyoxylate detoxification results in a diversion of glyoxylate to oxalate, with Hyp contributing up to ˜50% to urinary oxalate. Individuals with defects in HYPDH (Prodh2 gene) appear normal. Altogether, these data strongly support that the inhibition of HYPDH will be of therapeutic benefit to PH patients and patients with calcium oxalate kidney stone disease. Staufner et al., J Inherit Metab Dis 39(5):625-32 (2016).

The foregoing is illustrative of the present invention, and is not to be construed as limiting thereof. The invention is defined by the following claims, with equivalents of the claims to be included therein.

1. A method of treating kidney stones, comprising administering to a subject in need thereof, in combination, a hydroxyproline dehydrogenase (HYPDH) inhibitor, a glycolate oxidase (GO) inhibitor, and/or another agent for treatment of kidney stones.

2. The method of claim 1, wherein said HYPDH inhibitor is a compound of Formula I, a compound of Formula II, or a compound of Formula III: 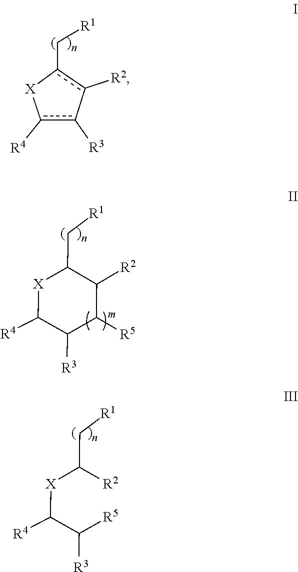
wherein:
X is O, S, NH, NMe or CR^{x}R^{y}, wherein R^{x} and R^{y} are each independently selected from H, alkyl or halo;
n is 0, 1, 2, 3, 4, 5 or 6;
m is 0, 1, 2, or 3;
R¹ is selected from the group consisting of: alkyl, alkenyl, alkynyl, aryl, halo, hydroxy, amine and carboxy;
R² is selected from the group consisting of: H, alkyl, hydroxy, amine, and ═O; or R² is R^{2a}R^{2b}, wherein R^{2a} and R^{2b} are each independently selected from alkyl and hydroxy;
R³ is selected from the group consisting of: H, hydroxy, amine, and ═O; or R³ is R^{3a}R^{3b}, wherein R^{3a} and R^{3b} are each independently selected from alkyl and hydroxy;
R⁴ is selected from the group consisting of: H, alkyl, and hydroxy; or R⁴ is R^{4a}R^{4b} wherein R^{4a} and R^{4b} are each independently selected from alkyl, hydroxy, and halo, wherein said alkyl may be unsubstituted or substituted 1, 2 or 3 times with hydroxy; and
each R⁵ is independently selected from the group consisting of: H, alkyl, hydroxy, amine, and ═O; or R⁵ is R^{5a}R^{5b} wherein R^{5a} and R^{5b} are each independently selected from alkyl and hydroxy; or R² and an adjacent R⁵ are taken together to form an aryl or heteroaryl,
or a pharmaceutically acceptable salt or prodrug thereof.

3. The method of claim 1, wherein said HYPDH inhibitor is a compound of Formula I: 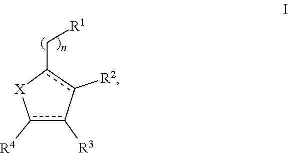
wherein:
X is S;
n is 0;
R¹ is selected from the group consisting of: alkyl, alkenyl, alkynyl, aryl, halo, hydroxy, amine and carboxy;
R² is selected from the group consisting of: H and lower alkyl;
R³ is selected from the group consisting of: hydroxy, amine, and ═O; or R³ is R^{3a}R^{3b}, wherein R^{3a} and R^{3b} are each independently selected from alkyl and hydroxy;
R⁴ is selected from the group consisting of: H and lower alkyl;
or a pharmaceutically acceptable salt or prodrug thereof.

4. The method of claim 1, wherein said HYPDH inhibitor is selected from the group consisting of: 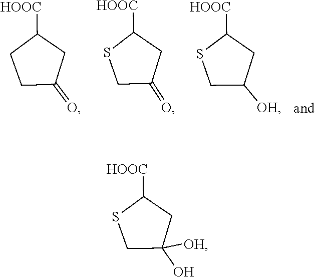
or a pharmaceutically acceptable salt or prodrug thereof.

5. The method of claim 1, wherein said HYPDH inhibitor is selected from the group consisting of: 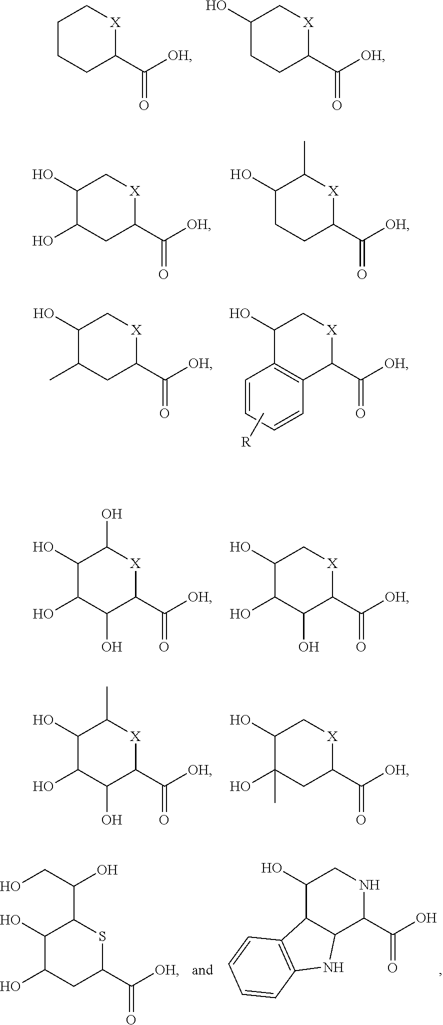
wherein:
X is NH, NMe, O or CH₂; and
R is H or OH,
or a pharmaceutically acceptable salt or prodrug thereof.

6. The method of claim 1, wherein said GO inhibitor is a compound of Formula IV or Formula V: 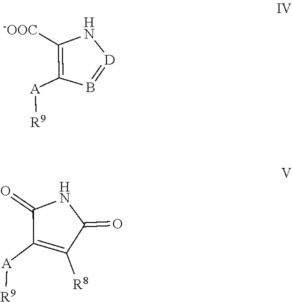
wherein:
A is CH₂ or S;
B is CH or N;
D is CH or N;
R⁸ is H or OH; and
R⁹ is aryl or heteroaryl, wherein said aryl or heteroaryl has two aromatic rings, which rings are fused or directly adjoining,
or a pharmaceutically acceptable salt or prodrug thereof.

7. The method of claim 1, wherein said GO inhibitor is a compound of Formula IV: 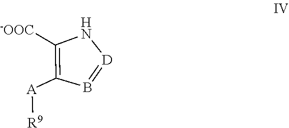
wherein:
A is CH₂ or S;
B is CH or N;
D is CH or N; and
R⁹ is aryl or heteroaryl, wherein said aryl or heteroaryl has two aromatic rings, which rings are fused or directly adjoining,
or a pharmaceutically acceptable salt or prodrug thereof.

8. The method of claim 6, wherein R⁹ is selected from the group consisting of: 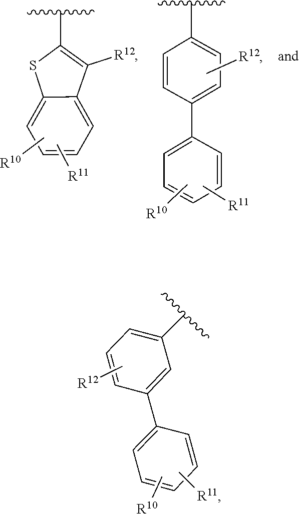
wherein R¹⁰, R¹¹ and R¹² are each independently selected from the group consisting of: H, alkyl, halo and haloalkyl,
or a pharmaceutically acceptable salt or prodrug thereof.

9. The method of claim 1, wherein said GO inhibitor is selected from the group consisting of: 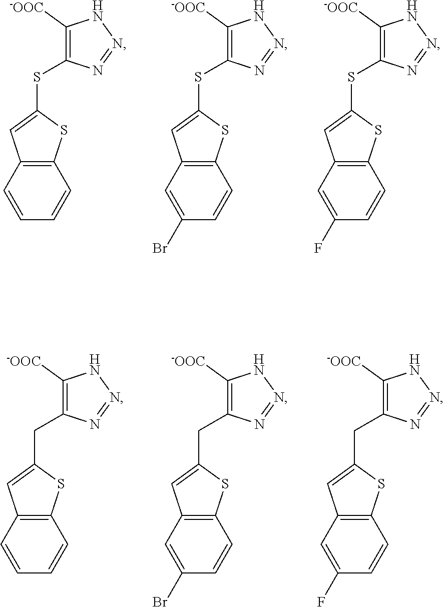 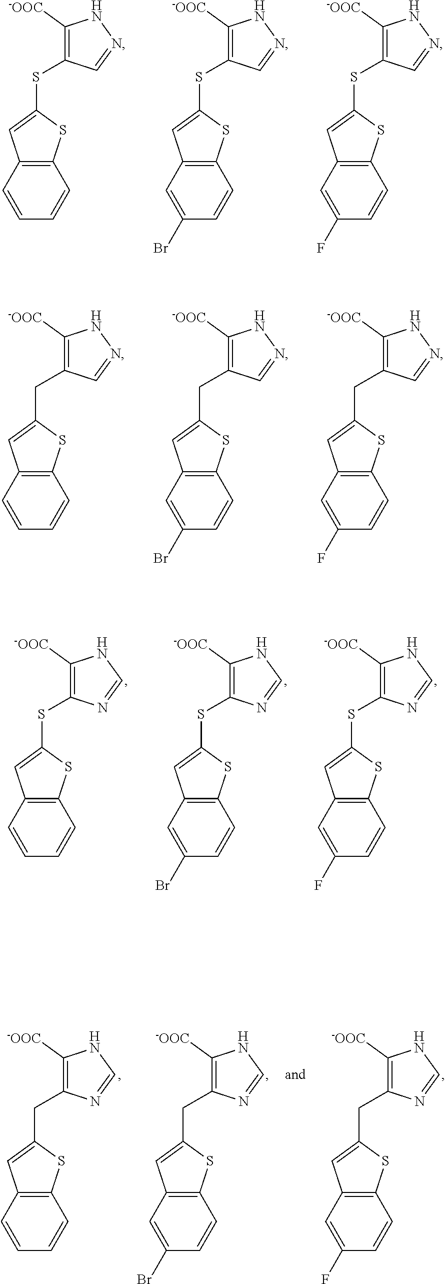
or a pharmaceutically acceptable salt thereof.

10. The method of claim 1, wherein said GO inhibitor is selected from the group consisting of: 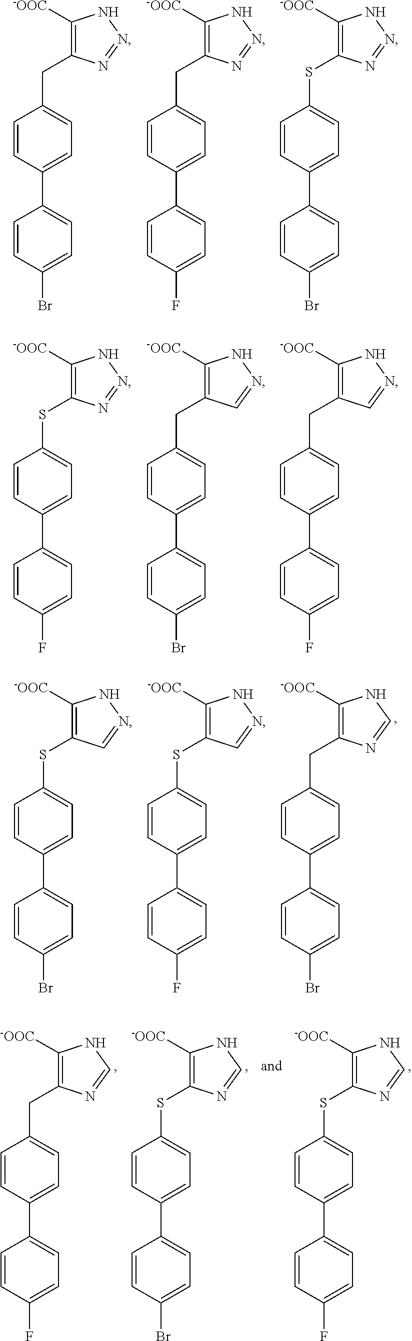
or a pharmaceutically acceptable salt thereof.

11. The method of claim 1, wherein said GO inhibitor is selected from the group consisting of: 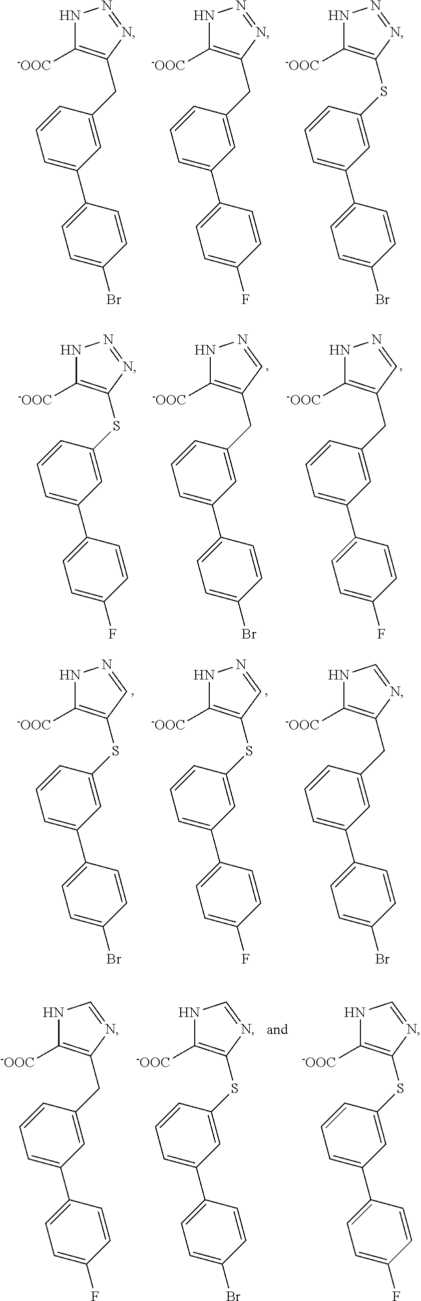
or a pharmaceutically acceptable salt thereof.

12. The method of claim 1, wherein said HYPDH inhibitor is administered in combination with said GO inhibitor.

13. The method of claim 1, wherein said method comprises administering a diuretic, a calcium oxalate crystallization inhibitor, an AGT cofactor or a kidney sodium glucose transporter inhibitor in combination with said HYPDH inhibitor.

14. A pharmaceutical composition comprising an HYPDH inhibitor, a GO inhibitor and/or another agent for the treatment of kidney stones.

15. The pharmaceutical composition of claim 14, wherein said composition is formulated for oral administration.

16. The pharmaceutical composition of claim 14, wherein said composition is a food product formulation.

17. The pharmaceutical composition of claim 14, wherein said composition is a capsule, cachet, lozenge, or tablet.

18. The method of claim 1, wherein said HYPDH inhibitor, GO inhibitor and/or another inhibitor of kidney stone formation are administered simultaneously.

19. The method of claim 1, wherein said HYPDH inhibitor, GO inhibitor and/or another inhibitor of kidney stone formation are administered sequentially.

20. The method of claim 1, wherein said subject is a human subject.

21. The method of claim 1, wherein said subject is a non-human animal subject.

22.-23. (canceled)
